(19) United States
(12) Patent Application Publication
(10) Pub. No.: US 2026/0041819 A1
(43) Pub. Date: Feb. 12, 2026

(54) TUNABLE, BIOCOMPATIBLE, NANOPARTICLE-CROSSLINKED HYALURONIC ACID-TYPE I COLLAGEN HYDROGEL USING DIELS-ALDER CLICK CHEMISTRY FOR REGENERATIVE MEDICINE APPLICATIONS

(71) Applicant: Bethany Almeida, Potsdam, NY (US)

(72) Inventor: Bethany Almeida, Potsdam, NY (US)

(73) Assignee: Clarkson University, Potsdam, NY (US)

(21) Appl. No.: 19/289,671

(22) Filed: Aug. 4, 2025

Related U.S. Application Data
(60) Provisional application No. 63/682,085, filed on Aug. 12, 2024.

(51) Int. Cl. A61L 27/52; A61L 27/48; A61L 27/54

(52) U.S. Cl. CPC A61L 27/52; A61L 27/48; A61L 27/54; A61L 2300/412; A61L 2300/414; A61L 2300/43; A61L 2300/624; A61L 2400/12

(57) ABSTRACT

The present disclosure provides a hydrogel composition for tissue engineering and regenerative medicine, including naturally derived polymers functionalized with furan groups and nanoparticles of poly(lactic-co-glycolic acid)-poly(ethylene glycol) copolymer functionalized with maleimide groups. Covalent crosslinking via a bioorthogonal Diels-Alder click reaction forms a tunable, biocompatible, and biodegradable three-dimensional network. The hydrogel composition mimics the extracellular matrix and offers adjustable mechanical properties, controlled biodegradation, and therapeutic agent delivery for applications such as cartilage regeneration and drug delivery.

### CROSS REFERENCE TO RELATED APPLICATION

The present application related and claims priority to Applicant's U.S. Provisional Application No. 63/682,085, filed Aug. 12, 2024, the entirety of which is hereby incorporated by reference.

### FIELD OF INVENTION

The present disclosure pertains to the field of tissue engineering and regenerative medicine, with a focus on biocompatible and biodegradable hydrogel scaffolds developed through advanced materials and covalent crosslinking mechanisms.

### BACKGROUND

Over time, hydrogel scaffolds have become widely utilized in tissue engineering and regenerative medicine due to their high water content and similarity to the native extracellular matrix. In vivo, the extracellular matrix provides both physical support and biochemical signals that influence cell behavior, proliferation, and differentiation. By entrapping cells within a hydrated polymer network, hydrogel scaffolds can safeguard cells during implantation and assist in tissue formation. However, finding the appropriate balance of mechanical strength, biochemical functionality, and degradation rate under physiological conditions continues to be a significant challenge.

Prior research on hydrogel materials has focused on balancing mechanical robustness with bioactivity and predictable biodegradation. The present approaches can be broadly categorized into two classes. Hydrogels derived from synthetic polymers offer tunable mechanical properties but typically lack inherent bioactivity, specific cell-adhesion sites, and predictable biodegradation profiles. On the other hand, naturally derived materials such as polysaccharides and proteins provide favorable cell interactions yet often exhibit insufficient mechanical integrity and rapid degradation. Chemical crosslinking strategies can improve structural robustness, but many employ reagents or reaction byproducts that are cytotoxic or induce adverse inflammatory responses. As a result, scaffolds that rely on conventional crosslinking methods frequently fall short of the stringent biocompatibility requirements for clinical translation.

Different approaches have been pursued to address the limitations of conventional hydrogel fabrication processes. These include fine-tuning polymer concentration, varying reaction conditions to control network architecture, and incorporating dynamic crosslinks to modulate viscoelastic behavior. However, achieving physiologically relevant stiffness, viscoelasticity, and biochemical presentation in a single platform is complicated by the disparate chemistries and processing conditions required for different polymer systems. As a result, many hydrogel formulations struggle to recapitulate the complex combination of cues found in native tissues, and thus underperform in preclinical and clinical evaluations.

In addition, recent efforts have explored the use of bioorthogonal covalent chemistries and the incorporation of particulate crosslinkers to modulate hydrogel structure without compromising cytocompatibility. However, a broadly adaptable methodology that systematically integrates natural polymers with non-toxic crosslinking agents and colloidal linkers under mild conditions has yet to be developed. This highlights the demand for a versatile platform that facilitates precise adjustment of polymer composition, crosslinker characteristics, and network formation kinetics to produce biocompatible, biodegradable hydrogels with physiologically relevant mechanical and biochemical properties for tissue engineering applications.

### SUMMARY

A hydrogel composition is disclosed that includes one or more naturally derived polymers modified with furan groups and nanoparticles made from a poly(lactic-co-glycolic acid)-poly(ethylene glycol) copolymer bearing maleimide groups. These components are covalently crosslinked through a bioorthogonal Diels-Alder click reaction, resulting in a three-dimensional hydrogel network. The naturally derived polymers can include hyaluronic acid and type I collagen, with the hyaluronic acid having an average molecular weight in the range of 500 kDa to 2 MDa and the type I collagen being sourced from bovine tendon. The nanoparticles are characterized by an average hydrodynamic diameter between 50 nm and 200 nm and a zeta potential from −25 mV to −5 mV. The ratio of furan groups to maleimide groups is adjustable between 1:1 and 1:10. The hydrogel composition can also include a therapeutic agent encapsulated within the nanoparticles, such as kartogenin, dexamethasone, or transforming growth factor-β. The resulting hydrogel displays a compressive modulus in the range of 0.5 kPa to 5 kPa. The Diels-Alder click reaction is performed at 37° C. and at a pH between 7.0 and 7.4. The poly(lactic-co-glycolic acid) component has a lactic acid to glycolic acid molar ratio of 50:50, and the polyethylene glycol has a molecular weight between 2 kDa and 5 kDa.

A method for fabricating such a hydrogel is also provided. The method involves functionalizing one or more naturally derived polymers with furan groups, preparing nanoparticles from a poly(lactic-co-glycolic acid)-poly(ethylene glycol) copolymer functionalized with maleimide groups, mixing the furan-functionalized polymers with the maleimide-functionalized nanoparticles, and covalently crosslinking the furan and maleimide groups via a bioorthogonal Diels-Alder click reaction to form the hydrogel network. The functionalization of hyaluronic acid is achieved by reacting hyaluronic acid with furfurylamine in the presence of 4-(4,6-dimethoxy-1,3,5-triazin-2-yl)-4-methylmorpholinium chloride (DMTMM), while type I collagen is functionalized by reacting type I collagen with furfurylglycidyl ether under basic pH conditions. Nanoparticle fabrication includes activating the carboxylic acid end groups of poly(lactic-co-glycolic acid) with 1-ethyl-3-(3-dimethylaminopropyl) carbodiimide (EDC) and N-hydroxysuccinimide (NHS), conjugating the activated polymer to amine-terminated poly(ethylene glycol)-maleimide, and forming nanoparticles by dissolving the copolymer in acetonitrile and introducing the copolymer into an aqueous phase via nanoprecipitation. The crosslinking reaction is carried out at 37° C. and at a pH between 7.0 and 7.4. The nanoparticles produced have an average hydrodynamic diameter between 50 nm and 200 nm. The hydrogel can further include a therapeutic agent encapsulated within the nanoparticles, such as kartogenin, dexamethasone, or transforming growth factor-β. The ratio of furan to maleimide groups is between 1:1 and 1:10, and the hydrogel exhibits a compressive modulus between 0.5 kPa and 5 kPa.

### BRIEF DESCRIPTION OF THE DRAWINGS

FIG. 1(A) is a schematic illustrating the functionalization of HA with a furan group.

FIG. 1(B) is a schematic illustrating the functionalization of Coll with a furan group.

FIG. 1(C) is a schematic illustrating the synthesis of PLGA-PEG-mal copolymer.

FIG. 1(D) is a schematic illustrating the fabrication of PLGA-PEG-mal NP via nanoprecipitation.

FIG. 1(E) is a schematic illustrating the formation of HA-NP-Coll hydrogel using Diels-Alder click chemistry.

FIG. 1(F) is a schematic illustrating the tunability of HA-PEG-Coll hydrogels with varying PEG concentrations and stiffness.

FIG. 1(G) is a schematic illustrating gelation and mechanical properties of HA-NP-Coll hydrogels.

### DETAILED DESCRIPTION

The present detailed description provides an in-depth explanation of embodiments, which pertain to the field of tissue engineering and regenerative medicine. Specifically, the subject matter relates to biocompatible and biodegradable hydrogel scaffolds formed using innovative combinations of materials and covalent crosslinking mechanisms. These scaffolds are designed to mimic the native extracellular matrix, offering tunable mechanical and biochemical properties for a wide range of applications, including but not limited to cartilage tissue engineering and drug delivery systems. The described approach leverages bioorthogonal Diels-Alder click chemistry and nanoparticle-based crosslinking to address longstanding challenges in hydrogel design, such as achieving physiologically relevant stiffness, bioactivity, and predictable biodegradation.

The examples and embodiments described herein are provided for illustrative purposes only and are not intended to limit the scope of the subject matter disclosed. Certain details, such as routine experimental techniques or well-understood principles in the field, may be omitted for clarity and brevity. Furthermore, various modifications, substitutions, or rearrangements of components, materials, or methods described herein may be made without departing from the spirit and scope of the subject matter, as defined by the appended claims. The disclosed subject matter is intended to encompass all such variations and equivalents that would be apparent to those skilled in the art.

Hydrogel scaffolds have emerged as promising materials in tissue engineering and regenerative medicine due to their ability to mimic the native extracellular matrix (ECM), which provides structural support and biochemical cues to cells. Despite their potential, conventional hydrogel systems face significant limitations. Synthetic polymer-based hydrogels offer tunable mechanical properties but lack bioactivity, cell-adhesion sites, and predictable biodegradation profiles. Conversely, naturally derived hydrogels exhibit favorable cell interactions but suffer from insufficient mechanical integrity and rapid degradation. Chemical crosslinking methods have been employed to enhance structural robustness, but many rely on cytotoxic reagents or produce harmful byproducts, compromising biocompatibility. Furthermore, achieving physiologically relevant stiffness, viscoelasticity, and biochemical functionality in a single hydrogel platform remains a challenge due to the disparate chemistries and processing conditions required for different polymer systems. These limitations hinder the ability of current hydrogel formulations to replicate the complex combination of cues found in native tissues, resulting in suboptimal performance in preclinical and clinical applications.

The present disclosure addresses these longstanding challenges by introducing a tunable, biocompatible, and biodegradable hydrogel scaffold that integrates naturally derived polymers with bioorthogonal Diels-Alder click chemistry and nanoparticle-based crosslinking. This approach combines hyaluronic acid (HA) and type I collagen (Coll), both functionalized with furan groups, with poly(lactic-co-glycolic acid)-poly(ethylene glycol) (PLGA-PEG) nanoparticles functionalized with maleimide groups. The Diels-Alder click reaction enables covalent crosslinking under mild, cell-friendly conditions, avoiding cytotoxic byproducts and ensuring biocompatibility. The use of nanoparticles as crosslinkers introduces a mechanism for enhancing mechanical properties while simultaneously enabling localized and sustained delivery of bioactive molecules, such as drugs or growth factors, encapsulated within the nanoparticles. This dual functionality addresses the need for both structural integrity and biochemical signaling in tissue engineering applications.

The hydrogel platform is highly tunable, allowing for precise adjustment of polymer concentrations, molecular weights, molar ratios between polymers and crosslinkers, and nanoparticle characteristics such as size and surface charge. This tunability enables the customization of physicochemical properties to meet the specific requirements of various regenerative medicine applications, such as cartilage tissue engineering or drug delivery systems. By combining the bioactivity and cell-adhesion properties of natural polymers with the mechanical robustness and controlled degradation afforded by nanoparticle crosslinking, the described hydrogel platform provides a versatile and clinically translatable solution to the limitations of conventional hydrogel systems.

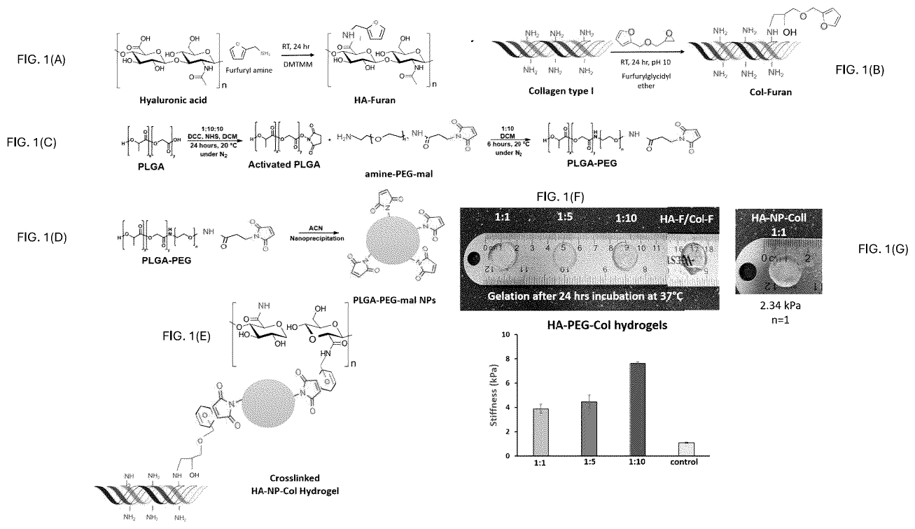

Referring to FIG. 1(A), the functionalization of hyaluronic acid (HA) with a furan moiety to yield HA-Furan is depicted. In some embodiments, this process involves reacting hyaluronic acid with furfurylamine in the presence of 4-(4,6-dimethoxy-1,3,5-triazin-2-yl)-4-methylmorpholinium chloride (DMTMM) as a coupling agent. The resulting HA-Furan serves as a hydrogel precursor, as the pendant furan groups enable bioorthogonal Diels-Alder click chemistry with maleimide-functionalized crosslinkers.

Accordingly, FIG. 1(B) illustrates the preparation of furan-modified type I collagen (Coll-Furan). Specifically, type I collagen is reacted with furfurylglycidyl ether via a ring-opening reaction under basic pH conditions. The furan-functionalized collagen introduces additional covalent crosslinking sites, thereby enhancing both the structural integrity and the bioactivity of the ensuing hydrogel network.

FIG. 1(C) illustrates the synthesis of a poly(lactic-co-glycolic acid)-poly(ethylene glycol) copolymer bearing terminal maleimide groups (PLGA-PEG-mal 104). In this process, the carboxylic acid end groups of PLGA are activated through treatment with 1-ethyl-3-(3-dimethylaminopropyl) carbodiimide (EDC) and N-hydroxysuccinimide (NHS), followed by conjugation to amine-terminated PEG-maleimide. As a result, the PLGA-PEG-mal 104 copolymer functions as the precursor for nanoparticle fabrication.

FIG. 1(D) demonstrates nanoprecipitation-mediated formation of PLGA-PEG-mal nanoparticles (PLGA-PEG-mal NP 105). The PLGA-PEG-mal 104 copolymer is dissolved in acetonitrile and subsequently introduced into an aqueous phase, resulting in self-assembly of nanoparticles. The exposed maleimide groups on the nanoparticle surface facilitate covalent crosslinking with the furan-functionalized polymers, while the PEG segments confer enhanced biocompatibility and colloidal stability.

Referring now to FIG. 1(E), the construction of the HA-NP-Coll hydrogel (HA-NP-Coll hydrogel 106) via Diels-Alder click chemistry is shown. The maleimide functionalities on the PLGA-PEG-mal nanoparticles (PLGA-PEG-mal NP 105) react efficiently with furan groups on both HA-Furan and Coll-Furan under mild conditions, yielding a covalently crosslinked hydrogel network. Importantly, this bioorthogonal reaction proceeds without generating cytotoxic byproducts, thereby ensuring cytocompatibility for tissue engineering applications.

FIG. 1(F) highlights the tunability of HA-PEG-Coll hydrogels (HA-PEG-Coll hydrogel 107) prepared using PEG-bis-maleimide as a crosslinker. The top panel demonstrates gelation and increasing stiffness as a function of PEG concentration; higher molar ratios of PEG-bis-maleimide yield correspondingly stiffer hydrogels. The bottom panel, by comparison to control hydrogels lacking PEG-bis-maleimide, confirms that covalent crosslinking substantially enhances mechanical properties.

FIG. 1(G) presents gelation kinetics and mechanical characterization of HA-NP-Coll hydrogels (HA-NP-Coll hydrogel 106). The left panel illustrates successful gel formation after 24 hours of incubation at 37° C. across varying HA-Furan to PLGA-PEG-mal nanoparticle (PLGA-PEG-mal NP 105) ratios. The right panel quantifies compressive moduli, demonstrating that nanoparticle-crosslinked hydrogels exhibit significantly higher stiffness compared to hydrogels formed without nanoparticles or maleimide-functionalized crosslinkers. These results validate the use of the Diels-Alder click reaction in generating robust hydrogel networks.

The hydrogel composition achieves a covalently crosslinked three-dimensional network through the bioorthogonal Diels-Alder click reaction between furan-functionalized naturally derived polymers and maleimide-functionalized nanoparticles. This specific crosslinking mechanism provides several advantages over conventional methods.

First, the use of the Diels-Alder click reaction ensures that the crosslinking occurs under mild, cell-friendly conditions (e.g., physiological temperature and pH), avoiding the generation of cytotoxic byproducts. This enhances the biocompatibility of the hydrogel, making the material suitable for tissue engineering and regenerative medicine applications where maintaining cell viability is of significant importance.

Second, the integration of nanoparticles as crosslinkers introduces a novel structural reinforcement mechanism. The nanoparticles, composed of a poly(lactic-co-glycolic acid)-poly(ethylene glycol) copolymer, enhance the mechanical robustness of the hydrogel while preserving the material's biodegradability. This design allows the hydrogel to offer adequate structural support during tissue regeneration and degrade in a controlled manner over time without leaving harmful residues.

Additionally, the covalent crosslinking between the furan and maleimide groups results in a stable hydrogel network with tunable mechanical properties. By adjusting the molar ratio of furan to maleimide groups, the stiffness and viscoelasticity of the hydrogel can be tailored to mimic the mechanical environment of specific tissues, thereby enhancing the functionality of the hydrogel in various regenerative medicine applications.

The combination of naturally derived polymers, such as hyaluronic acid and type I collagen, with the nanoparticle-based crosslinking strategy further ensures that the hydrogel mimics the native extracellular matrix. This facilitates cell adhesion, proliferation, and differentiation, which are necessary for promoting tissue regeneration effectively.

What is claimed is:

1. A hydrogel composition comprising:
one or more naturally derived polymers functionalized with furan groups;
nanoparticles comprising a poly(lactic-co-glycolic acid)-poly(ethylene glycol) copolymer functionalized with maleimide groups;
wherein the furan groups and the maleimide groups are covalently crosslinked via a bioorthogonal Diels-Alder click reaction to form a three-dimensional hydrogel network.

2. The hydrogel composition of claim 1, wherein said one or more naturally derived polymers comprise:
hyaluronic acid; and
type I collagen.

3. The hydrogel composition of claim 2, wherein said hyaluronic acid has an average molecular weight between 500 kDa and 2 MDa.

4. The hydrogel composition of claim 2, wherein said type I collagen is derived from a bovine tendon source.

5. The hydrogel composition of claim 1, wherein said nanoparticles have an average hydrodynamic diameter between 50 nm and 200 nm.

6. The hydrogel composition of claim 5, wherein said nanoparticles have a zeta potential between −25 mV and −5 mV.

7. The hydrogel composition of claim 1, wherein said molar ratio of furan groups to maleimide groups is between 1:1 and 1:10.

8. The hydrogel composition of claim 1, wherein the composition further comprises:
a therapeutic agent encapsulated within said nanoparticles.

9. The hydrogel composition of claim 8, wherein said therapeutic agent is selected from the group consisting of:
kartogenin;
dexamethasone; and
transforming growth factor-β.

10. The hydrogel composition of claim 1, wherein said hydrogel exhibits a compressive modulus between 0.5 kPa and 5 kPa.

11. The hydrogel composition of claim 1, wherein said bioorthogonal Diels-Alder click reaction is conducted:
at 37° C.; and
at a pH of between 7.0 and 7.4.

12. The hydrogel composition of claim 1, wherein said poly(lactic-co-glycolic acid) comprises a lactic acid to glycolic acid molar ratio of 50:50.

13. The hydrogel composition of claim 1, wherein said polyethylene glycol has a molecular weight between 2 kDa and 5 kDa.

14. A method for fabricating a hydrogel, the method comprising:
functionalizing one or more naturally derived polymers with furan groups;
fabricating nanoparticles comprising a poly(lactic-co-glycolic acid)-poly(ethylene glycol) copolymer functionalized with maleimide groups;
combining the furan-functionalized polymers and the maleimide-functionalized nanoparticles; and
covalently crosslinking the furan groups and the maleimide groups via a bioorthogonal Diels-Alder click reaction to form a three-dimensional hydrogel network.

15. The method of claim 14, wherein the step of functionalizing one or more naturally derived polymers with furan groups comprises:
reacting hyaluronic acid with furfurylamine in the presence of 4-(4,6-dimethoxy-1,3,5-triazin-2-yl)-4-methylmorpholinium chloride (DMTMM).

16. The method of claim 14, wherein the step of functionalizing one or more naturally derived polymers with furan groups comprises:
reacting type I collagen with furfurylglycidyl ether under basic pH conditions.

17. The method of claim 14, wherein the step of fabricating nanoparticles comprises:
activating carboxylic acid end groups of poly(lactic-co-glycolic acid) with 1-ethyl-3-(3-dimethylaminopropyl) carbodiimide (EDC) and N-hydroxysuccinimide (NHS); and
conjugating the activated poly(lactic-co-glycolic acid) to amine-terminated poly(ethylene glycol)-maleimide.

18. The method of claim 17, wherein the step of fabricating nanoparticles further comprises:
dissolving the poly(lactic-co-glycolic acid)-poly(ethylene glycol)-maleimide copolymer in acetonitrile and introducing the solution into an aqueous phase to form nanoparticles by nanoprecipitation.

19. The method of claim 14, wherein the step of covalently crosslinking the furan groups and the maleimide groups via a bioorthogonal Diels-Alder click reaction is conducted at 37° C. and at a pH of between 7.0 and 7.4.

20. The method of claim 14, wherein the nanoparticles have an average hydrodynamic diameter between 50 nm and 200 nm.

21. The method of claim 14, wherein the hydrogel further comprises a therapeutic agent encapsulated within the nanoparticles.

22. The method of claim 21, wherein the therapeutic agent is selected from the group consisting of:
kartogenin;
dexamethasone; and
transforming growth factor-β.

23. The method of claim 14, wherein the molar ratio of furan groups to maleimide groups is between 1:1 and 1:10.

24. The method of claim 14, wherein the hydrogel exhibits a compressive modulus between 0.5 kPa and 5 kPa.
